(19) United States
(12) Patent Application Publication
(10) Pub. No.: US 2025/0123501 A1
(43) Pub. Date: Apr. 17, 2025

(54) ILLUMINATED CONTACT LENS SYSTEM

(71) Applicant: Alcon Inc., Fribourg (CH)

(72) Inventors: Yu Yan, Irvine, CA (US); John Park, Irvine, CA (US); Qing Xiang, Irvine, CA (US)

(21) Appl. No.: 18/911,759

(22) Filed: Oct. 10, 2024

Related U.S. Application Data
(60) Provisional application No. 63/589,800, filed on Oct. 12, 2023.

(51) Int. Cl. G02C 11/04; G02C 7/04; G02C 11/00

(52) U.S. Cl. CPC G02C 11/04; G02C 7/04; G02C 11/10; G02C 2202/06

(57) ABSTRACT

An illuminated contact lens system for illuminating an interior of a patient's eye includes a contact lens, an external light source, and a light engine. The lens refracts directed light into the eye. The light source emits the directed light toward the eye. The light engine transmits electronic control signals that cause the light source to emit the directed light. A prism array connected to an annular tube containing the light source may optionally shape the directed light into a conical distribution pattern. A control board may drive the light source in response to the electronic control signals, and may be optionally used as a proximity sensor. A method includes receiving the electronic control signals from the light engine via the control board, illuminating the light source, and emitting the directed light from the toward the eye.

### RELATED APPLICATION

This application claims benefit under 35 U.S.C. § 119 (e) of U.S. Provisional Application Ser. No. 63/589,800, entitled “ILLUMINATED CONTACT LENS SYSTEM” Attorney's Docket PAT059189-US-PSP, filed Oct. 12, 2023, which is incorporated herein by reference.

### INTRODUCTION

The present disclosure relates to ophthalmic lenses and associated contact lens-based lighting control methodologies for illuminating the interior structure of a patient's eye.

Directed lighting of the vitreous chamber of a patient's eye allows a surgeon to properly visualize the eye's internal anterior and posterior anatomy. Vitreoretinal surgeries, for instance, often call for the use of directed or diffused light depending on the particular surgical task that is being performed. In order provide such lighting, the surgeon forms a small incision in the sclera. A lighting tool is then inserted through the incision via a transscleral cannula. Based on the particular configuration of the lighting tool, the surgeon is able to illuminate the posterior chamber using directed light, e.g., through an inserted endoilluminator probe, or using diffuse light, for instance using a fiberoptic chandelier-type illuminator.

As appreciated in the art, light provided by current ophthalmic illumination systems is generally not widely distributed within the patient's eye. Instead, the light emitted by the above-summarized lighting tools tends to be concentrated nearest the central retina or macula area. During certain ophthalmic procedures, however, it is desirable to illuminate the retina's outer peripheral area or skirt, such as when treating posterior vitreous detachments or removing problematic floaters. It is also desirable at times to illuminate anterior areas of the eye, e.g., for foreign bodies or other objects of interest.

### SUMMARY

Disclosed herein are an illuminated contact lens system and a related control methodology for illuminating areas of interest within a patient's eye during an ophthalmic procedure. The lens system described herein, which directly or indirectly integrates an external lighting device with a patient-wearable contact lens, is intended to address the above-noted problems, such as but not limited to light concentration nearest the center retina area. The disclosed lens system is also non-invasive. That is, unlike cannula-based internal lighting techniques, the lens system of the present disclosure can illuminate the posterior chamber and anterior sections of the eye from outside of the eye, without precluding the possible use of the present solutions with endoilluminator probes or chandeliers. This beneficial feature in turn facilitates pre-surgical and post-surgical visualization efforts as well as in-office examinations of the patient's ocular health.

In accordance with an aspect of the disclosure, the illuminated contact lens system may include a contact lens that is wearable on a patient's eye. The lens system also includes an external light source and a light engine. The contact lens is configured to refract directed light from the external light source into the eye, such as onto a peripheral retina area of the eye in one or more implementations. The light engine in this particular embodiment, e.g., hardware and associated software of an associated ophthalmic surgical console, is configured to transmit electronic control signals to the external light source to cause the external light source to emit the directed light toward the contact lens.

The lens system may include an annular tube and a prism array. The annular tube in one or more embodiments contains the light source therewithin. The prism array is connected to the annular tube and is configured to shape the directed light from the light source into a conical distribution pattern. This occurs when the prism array is illuminated by the directed light from the light source. The light source may be optionally positioned and oriented relative to the contact lens such that the peripheral retina area is illuminated via total internal reflection (TIR) within the annular tube.

In some implementations, a control board in communication with the light source is configured to drive the light source in response to the electronic control signals from the light engine. The control board in one or more embodiments may include an antenna operable for wirelessly receiving the electronic control signals from the light engine.

The control board may also be configured as a proximity sensor. The proximity sensor in such an embodiment is operable for detecting when the light source is separated from an external surface of the contact lens by a predetermined standoff distance, and for communicating an electronic proximity signal to the light engine that is indicative of the light source having reached the standoff distance.

The light source may include a light-emitting diode array having an adjustable setting that is responsive to the electronic control signals. The adjustable setting could include at least one of a color, a temperature, or a brightness level of the light-emitting diode array.

In one or more embodiments, the light source is operatively connected to the contact lens to form an illuminated lens assembly.

An aspect of the disclosure includes a surgical console in communication with the light source and having a processor and a computer-readable storage medium. The light engine in this embodiment includes the processor and the computer-readable storage medium.

In another aspect of the disclosure, an illuminated lens system includes a contact lens configured to refract directed light into a patient's eye, and a light source configured to emit the directed light toward the eye in response to electronic control signals from a light engine. The light source includes a plurality of light-emitting diodes (LEDs) arranged in a ring. As part of this optional construction, an annular tube is connected to the contact lens and encloses the light source therewithin. A prism array is connected to the annular tube and configured to shape the directed light into a conical distribution pattern when the prism array is illuminated by the light source, thereby illuminating an interior of the eye.

Also disclosed herein is a method for illuminating an interior of a patient's eye. The method may include receiving electronic control signals from a light engine via a control board having a light source, wherein the light source is mounted to a surface of the control board. The method also includes illuminating the light source in response to the electronic control signals from the light engine, and emitting directed light from the light source toward a contact lens arranged on the eye.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 is an illustration of an illuminated contact lens system constructed in accordance with an aspect of the present disclosure.

FIG. 2 is an illustration of the lens system shown in FIG. 1 according to a possible embodiment.

FIG. 3 is a schematic illustration of an alternative embodiment of the lens system shown in FIG. 2.

FIG. 4 is a flowchart describing a method for illuminating an interior of a patient's eye as described in detail below.

The solutions of the present disclosure may be modified or presented in alternative forms. Representative embodiments are shown by way of example in the drawings and described in detail below. However, inventive aspects of this disclosure are not limited to the disclosed embodiments. Rather, the present disclosure is intended to cover alternatives falling within the scope of the disclosure as defined by the appended claims.

### DETAILED DESCRIPTION

Embodiments of the present disclosure are described herein. It is to be understood, however, that the disclosed embodiments are merely examples and other embodiments can take various and alternative forms. The figures are not necessarily to scale. Some features could be exaggerated or minimized to show details of particular components. Therefore, specific structural and functional details disclosed herein are not to be interpreted as limiting, but merely as a representative basis for teaching one skilled in the art to variously employ the present disclosure.

As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

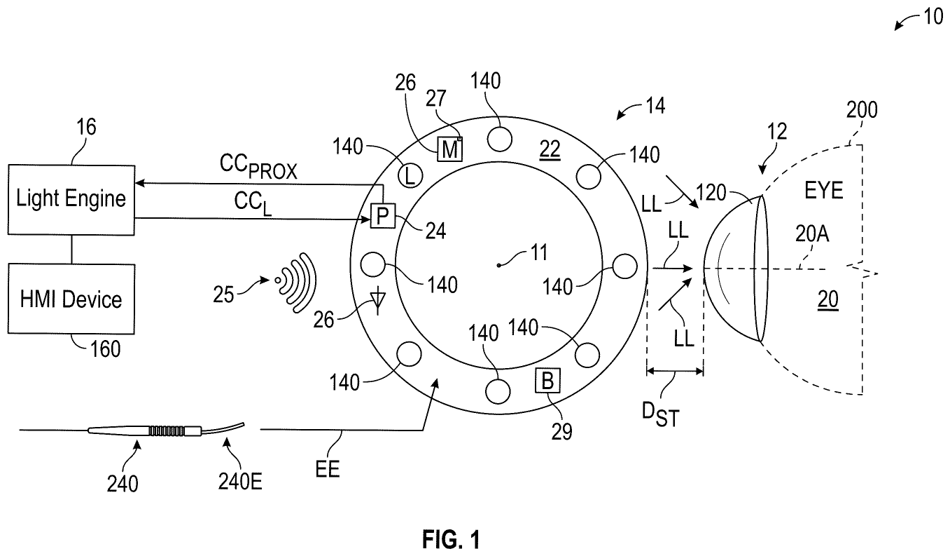

Referring to the drawings, wherein like reference numbers refer to like components, and beginning with FIG. 1, an illuminated contact lens system 10 is shown relative to a patient's eye 20. The lens system 10 in accordance with the present disclosure includes a contact lens 12 and an external light source 14, also referred to herein as a ring illuminator. As used herein, “external” means “outside of the eye 20”, i.e., in contrast to insertable/invasive light tools or probes of the types summarized above. The lens system 10 also includes a light engine 16 in some embodiments, or the lens system 10 is at least in wired or wireless communication with the light engine 16, e.g., remotely via a radio frequency (RF) signal 25.

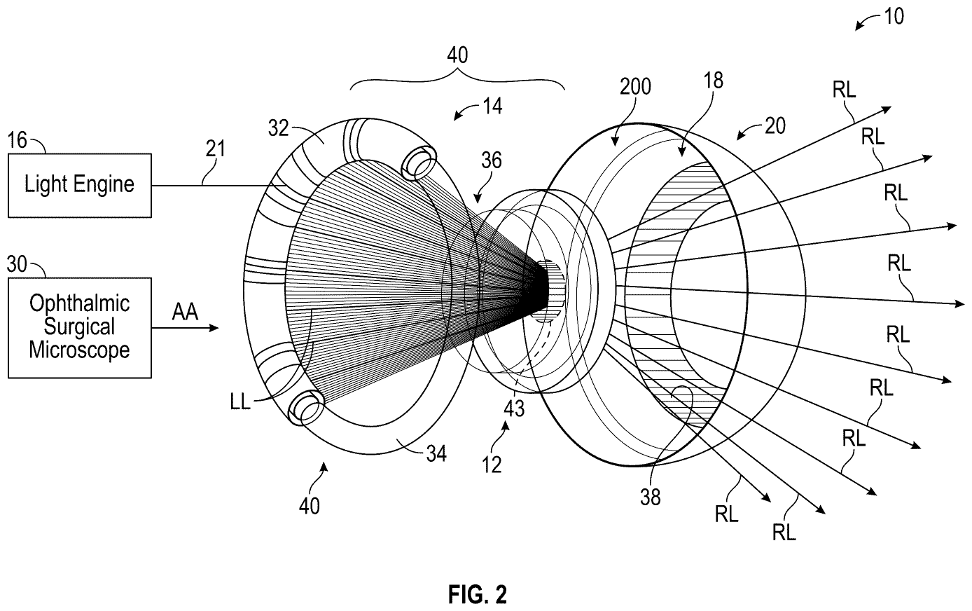

The contact lens 12, which is wearable on a cornea (not shown) of a patient's eye 20 as appreciated in the art, is configured to refract the directed light (LL) in an annular illumination pattern 38 into the eye 20, such as but not limited to a peripheral retina area 18 located in a posterior chamber 200 of the eye 20 as depicted in FIG. 2. Other implementations could illuminate anterior portions of the eye 20, and therefore the present teachings are not limited to illumination of the posterior chamber 200. The light engine 16 for its part is configured to transmit electronic control signals (CC_{L}) to the external light source 14 to thereby cause the external light source 14 to emit the directed light (LL) toward the eye 20.

The external light source 14 in accordance with an aspect of the disclosure may be positioned and oriented relative to the contact lens 12 such that the peripheral retina area 18 (FIG. 2) in this particular non-limiting example is illuminated via total internal reflection (TIR) within an annular tube 32 as described herein. That is, the contact lens 12 and the external light source 14 could be arranged to ensure that at least some of the directed light (LL) passes through the contact lens 12 at the critical angle needed for achieving TIR.

Embodiments of the external light source 14 could include, by way of example and not of limitation, a plurality of light-emitting diodes (LEDs) 140, e.g., an LED array. For instance, the external light source 14 could include red, green, and blue (RGB) LED array and at least one near-infrared (near-IR) LED in a possible embodiment. In other embodiments, the external light source 14 could include halogen bulbs, optical fibers, or other application-suitable lighting devices in lieu of the LEDs 140, or possibly an endoilluminator 240 having a tip 240E that is insertable into or connected to the external light source 14. Once inserted into the external light source 14, the endoilluminator 240 would emit light (EE) into the external light source 14 via its tip 240E, with the external light source 14 thereafter bending or shaping the light (EE) into the directed light (LL) as described below.

In keeping with the non-limiting embodiment of FIG. 1 in which the external light source 14 includes the plurality of LEDs 140, each respective LED 140 may have an adjustable setting that is responsive to the electronic control signals (CC_{L}) from a control board 22. The control board 22 is depicted in FIG. 1 as an annular printed circuit board having a center axis 11 and multiple surface-mounted components including a processor (P) 24, memory (M) 26, an RF antenna 26, and possibly a battery (B) 29. In other implementations, the control board 22 could have another application-suitable shape, and could be miniaturized and integrated with the structure of the lighting source 14, for instance as a microelectromechanical system (MEMS) circuit, a microchip, or a system-on-a-chip (SoC).

The adjustable settings could include at least one of a color, a temperature, or a brightness level of the LEDs 140 in some embodiments, e.g., cool, warm white, monochromatic, RGB, etc., with the adjustable settings possibly being user-selectable via the light engine 16 and a human-machine interface (HMI) device 160. Being individually-addressable, the exemplary LEDs 140 could be individually controlled using the same or different adjustable settings depending on the configuration, e.g., via operation of the light engine 16 when the light engine 16 is an integral part of an ophthalmic surgical console of the type appreciated in the art.

Still referring to FIG. 1, the control board 22 is configured to electrically drive the external light source 14 in response to the electronic control signals (CC_{L}) from the light engine 16. To that end, the RF antenna 26 could be used to wirelessly receive the electronic control signals (CC_{L}) from the light engine 16. As contemplated herein, the control board 22 is programmed in software and equipped in hardware, i.e., “configured”, to execute computer-readable instructions from non-volatile portions of its memory 27 using the processor 24. The memory 27 is configured as a computer-readable storage medium, and thus includes application-specific amounts of tangible non-transitory memory, e.g., optical, magnetic, flash, or other types of read only memory, along with application-sufficient amounts of random-access memory, electrically-erasable programmable read only memory, etc.

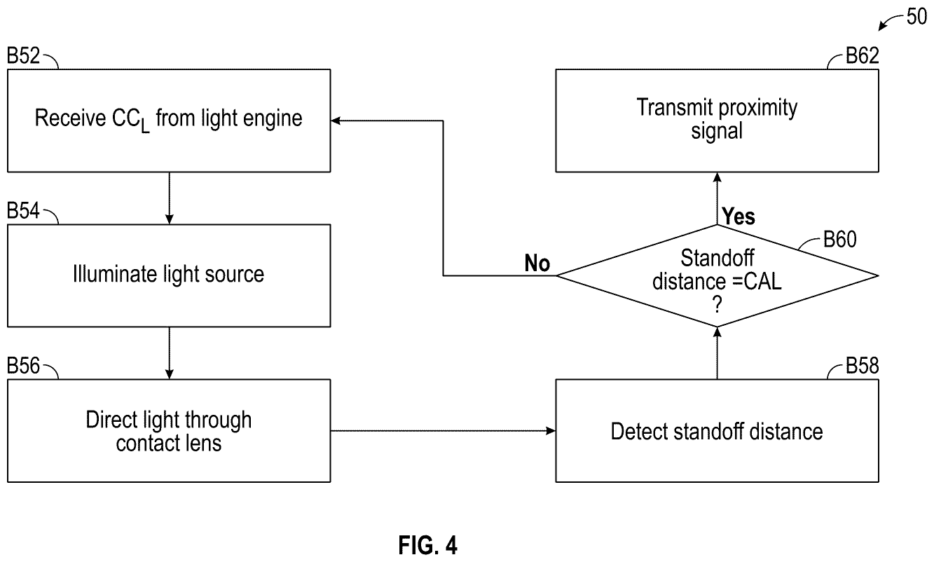

The processor 24 in turn may be constructed from various combinations of Application Specific Integrated Circuit(s) (ASICs), Field-Programmable Gate Arrays (FPGAs), electronic circuits, central processing units, microprocessors, and the like. Non-transitory components of the memory 27 record or store computer-readable instructions for controlling operation of the ophthalmic lens system 10 of FIG. 1, including an instruction set 27 for performing a method 50, an embodiment of which is depicted in FIG. 4 and described in detail below.

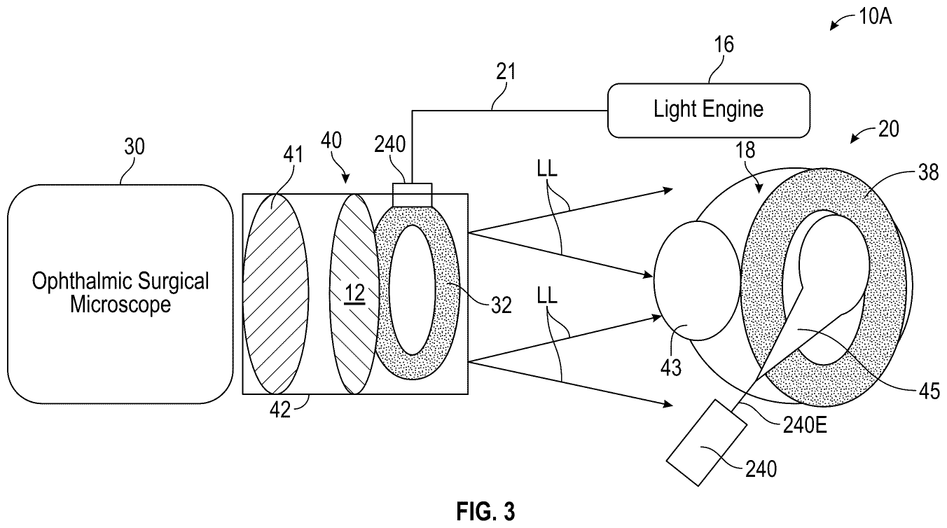

The control board 22 of FIG. 1 in some implementations may be configured as a proximity sensor operable for detecting when the external light source 14 is separated from an external surface 120 of the contact lens 12 by a predetermined standoff distance (D_{ST}), and for communicating an electronic proximity signal (CC_{PROX}) to the light engine 16 or another remote device that is indicative of the external light source 14 having reached the standoff distance (D_{ST}). Proximity detection within the scope of the disclosure could be performed by measuring a time-of-flight (TOF) of a modulated or pulsed light from the external light source 14. As illustrated in FIG. 1 for clarity, the external light source 14 is shown in a plan view while the eye 20 is depicted in side view. In actual use, however, the optical axis 20A of the eye 20 is coaxially-aligned with the center axis 11 of the external light source 14, with such an orientation best shown in FIGS. 2 and 3.

Referring now to FIG. 2, the illuminated contact lens system 10 in accordance with one or more embodiments includes the above-noted annular tube 32, which in turn contains the external light source 14 therewithin. The annular tube 32 may be constructed of molded plastic, glass, or another application-suitable transparent or translucent material. The light engine 16 is shown in a possible construction as being connected via an electrical cable 21 to the external light source 14. As part of such an implementation, a prism array 34, e.g., a microprism array or a mirror array, may be connected to or formed integrally with the annular tube 32. Unlike a typical diffuser panel, the prism array 34 of FIG. 2 is configured to shape light from the external light source 14 into the directed light (LL) and through the pupil 43 of the eye 20, e.g., as a conical distribution pattern 36 as shown. The external light source 14 could also be integrated into structure of an ophthalmic microscope 30 arranged on an optical axis (AA) as shown.

The external light source 14, the annular tube 32, and the prism array 34 illustrated in FIG. 2 collectively form a ring illuminator 40 within the scope of the present disclosure. The ring illuminator 40 can be used to illuminate desired internal anatomy of the eye 20, e.g., the peripheral retina area 18 of the eye 20, anterior anatomy, etc., as shown via the annular illumination pattern 38. The annular illumination pattern 38 is particularly useful, for instance, during vitrectomy surgeries. The external light source 14 in this embodiment could optionally include the endoilluminator 240 of FIG. 1, which in turn could be inserted into the annular tube 32. The annular tube 32 in this example would function as a light guide. The particular geometry of the annular tube 32 in such an embodiment is configured to refract and redirect any light emitted from the tip 240E into the eye 20 as indicated by light rays RL, and to thus generate the annular illumination pattern 38 on the peripheral retina area 18.

As shown in FIG. 3, the illuminated contact lens system 10 of FIG. 1 is shown in an alternative arrangement as a lens system 10A, in which the contact lens 12 is integrally formed with or physically attached to the remaining structure of the ring illuminator 40. In one or more implementations, the lens system 10A could be operatively connected to or integrated with the contact lens 12 to form an illuminated lens assembly 42, possibly assisted by a refracting lens 41 positioned adjacent the contact lens 12.

In this embodiment, the endoilluminator 240 of FIG. 1 could be energized via the light engine 16 to feed light (EE of FIG. 2) into the annular tube 32 of the ring illuminator 40. The directed light (LL) is then emitted toward the eye 20, at least some of which will pass through the pupil 43 and onto peripheral retina area 18 as the annular illumination pattern 38. In a possible surgical use, a surgeon could simultaneously employ an endoilluminator 240 within the eye 20, with the tip 240E of such an endoilluminator 240 emitting a concentrated light beam 45 radially within the annular illumination pattern 38 as shown.

As with the exemplary embodiment of FIG. 2, the ring illuminator 40 could be constructed as a tube-shaped light guide of molded plastic, with the optimized prism array 34 of FIG. 2 set at one side facing the contact lens 12 and a specific shape set on the opposite side to generate uniform light at a wide angle for distribution into the eye 20, rather than the narrow field-of-view provided by traditional illumination techniques. Other possible solutions include a spatial light modulation (SLM) ring structure, glass, a classic lens, and/or a mirror configuration.

Referring now to FIG. 4, the method 50 in accordance with an embodiment of the disclosure is organized into discrete logic blocks. Each logic block in turn represents a particular step, function, or subprocess that is to be performed using the ophthalmic lens system 10 of FIG. 1 to illuminate the peripheral retina area 18 of the eye 20 shown in FIGS. 1-3.

Commencing with block B52, the method 50 includes receiving the electronic control signals (CC_{L}) from the light engine 16 via the control board 22 having the external light source 14. As noted above, in some embodiments the external light source 14 may be mounted to a surface of the control board 22, e.g., as surface-mounted components. Receipt of the electronic control signals (CC_{L}) could be accomplished using the RF antenna 26 of FIG. 2 such that the illuminated contact lens system 10 is characterized by an absence of physical transfer conductors between the control board 22 and the light engine 16. Alternatively, a hard-wired connection could be established between the control board 22 and the light engine 16 using physical transfer conductors in different implementations. The method 50 proceeds to block B54 once the electronic control signals (CC_{L}) have been received.

Block B54 includes illuminating the external light source 14 of FIGS. 1-3 in response to the electronic control signals (CC_{L}) from the light engine 16. For example, the electronic control signals (CC_{L}) could individually address multiple LEDs 140 of FIG. 1 in a possible approach, e.g., as voltage signals commanding illumination in a particular color, color temperature, brightness, intensity, etc. In embodiments for which the external light source 14 is uniformly illuminated, the electronic control signals (CC_{L}) could themselves encompass drive signals that directly illuminate the external light source 14 as a whole. The method 50 then proceeds to block B56.

Block B56 includes directing the light into the eye 20, which in this exemplary instance occurs through the intervening contact lens 12. Within the scope of the disclosure, this may entail emitting the directed light (LL) from the external light source 14 toward the contact lens 12, the latter being arranged on the eye 20 of FIGS. 1-3. The contact lens 12 in this and other embodiments is configured to refract the directed light (LL) onto the peripheral retina area 18 (see FIG. 2). Some light could be directed outside of the contact lens 12 in one or more embodiments.

In one or more implementations, emitting the directed light (LL) from the external light source 14 toward the eye 20 includes illuminating the above-described prism array 34 of FIG. 2 with the directed light (LL). Such a prism array 34 could be connected to or formed integrally with the annular tube 32 containing the external light source 14 therein. In such an implementation, block B56 could include shaping the directed light (LL) into the conical distribution pattern 36 of FIG. 2 using the prism array 34. The method 50 then proceeds to block B58.

Block B58 of FIG. 4, which is optional within the scope of the method 50, may entail detecting the standoff distance (D_{ST}) of FIG. 1. For example, block B58 could include detecting, via the control board 22, when the external light source 14 is separated from the external surface 120 of the contact lens 12 of FIG. 1 by the predetermined standoff distance (D_{ST}). A suitable approach for detecting the standoff distance (D_{ST}) includes temporarily emitting the direct light (LL) as modulated or pulsed light for this purpose and tracking time-of-flight (TOF). The method 50 proceeds to block B60 once the processor 24 has ascertained the standoff distance (D_{ST}).

At block B60, the processor 24 may compare the standoff distance (D_{ST}) from block B58 to a calibrated distance (“CAL”), e.g., using a comparator circuit or associated logic. The method 50 then proceeds to block B52 when the standoff distance (D_{ST}) is less than the calibrated distance, and to block B60 in the alternative when the standoff distance (D_{ST}) exceeds the calibrated distance.

Block B62 includes transmitting the electronic proximity signal (CC_{PROX}) of FIG. 1 to the light engine 16 or to another device as a possible control action. The electronic proximity signal (CC_{PROX}) is indicative of the external light source 14 having reached the predetermined standoff distance (D_{ST}) detected in block B58. In response to receipt of the electronic proximity signal (CC_{PROX}), the light engine 16 could possibly perform a response action, e.g., sounding an audible alarm and/or illuminating a warning light, or possibly pulsing or strobing the directed light (LL) to alert the surgeon.

As will be appreciated by those skilled in the art in view of the foregoing disclosure, the illuminated contact lens system 10 or 10A of FIGS. 1-3 provide a myriad of benefits when used to visualize the internal anatomy of the eye 20. In a surgical use in which the illuminated contact lens system 10 or 10A is used in conjunction with an insertable light probe, e.g., the endoilluminator 240, the surgeon could perform surgery using one fewer cannula. This in turn frees up one of the surgeon's hands to perform a different task.

As shown in FIG. 3, for example, the ring illuminator 40 could be used to create the annular illumination pattern 38 on the peripheral retina area 18 in a possible approach. At the same time the endoilluminator 240 could be inserted and used create the concentrated light beam 45 radially within the annular illumination pattern 38. Alternatively, the surgeon could use the ring illuminator 40 for lighting the peripheral retina area 18 and one hand to manipulate a surgical tool, e.g., a laser device, vitrector, or other surgical tool as needed. Non-surgical uses could likewise be contemplated, for which the surgeon could use the lens system 10 or 10A without penetrating into the eye 20. These and other possible attendant benefits thus would follow from the disclosed solutions.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

1. An illuminated contact lens system, comprising:
a contact lens configured to be worn on a patient's eye, and to refract directed light into the eye;
an external light source configured to emit the directed light toward the eye; and
a light engine configured to transmit electronic control signals to the external light source to thereby cause the external light source to emit the directed light toward the eye, thereby illuminating an interior of the eye.

2. The lens system of claim 1, further comprising:
an annular tube containing the external light source therewithin; and
a prism array connected to the annular tube, wherein the prism array is configured to shape the directed light from the external light source into a conical distribution pattern when the prism array is illuminated by the directed light from the external light source.

3. The lens system of claim 2, wherein the external light source is positioned and oriented relative to the contact lens such that a peripheral retina area of the interior of the eye is illuminated via total internal reflection within the annular tube.

4. The lens system of claim 1, further comprising:
a control board in communication with the external light source, wherein the control board is configured to drive the external light source in response to the electronic control signals from the light engine.

5. The lens system of claim 4, wherein the control board includes an antenna operable for wirelessly receiving the electronic control signals from the light engine.

6. The lens system of claim 4, wherein the control board is configured as a proximity sensor that is operable for detecting when the external light source is separated from an external surface of the contact lens by a predetermined standoff distance, and for communicating an electronic proximity signal to the light engine that is indicative of the light source having reached the standoff distance.

7. The lens system of claim 1, wherein the external light source includes a light-emitting diode array having an adjustable setting that is responsive to the electronic control signals, and wherein the adjustable setting includes at least one of a color, a temperature, or a brightness level of the light-emitting diode array.

8. The lens system of claim 1, wherein the external light source is operatively connected to the contact lens to form an illuminated lens assembly.

9. The lens system of claim 1, further comprising:
a surgical console in communication with the external light source and having a processor and a computer-readable storage medium, wherein the light engine includes the processor and the computer-readable storage medium.

10. An illuminated contact lens system, comprising:
a contact lens configured to refract directed light into a patient's eye;
an external light source configured to emit the directed light toward the eye in response to electronic control signals from a light engine, wherein the light source includes a plurality of light-emitting diodes (LEDs) arranged in a ring;
an annular tube connected to the contact lens and enclosing the external light source therewithin; and
a prism array connected to the annular tube and configured to shape the directed light into a conical distribution pattern when the prism array is illuminated by the light source, thereby illuminating an interior of the eye.

11. The lens system of claim 10, wherein the external light source is positioned and oriented relative to the contact lens such that the interior of the eye is illuminated via total internal reflection.

12. The lens system of claim 10, further comprising:
a control board in communication with the light engine and the LEDs, wherein the control board is configured to drive the LEDs in response to the electronic control signals from the light engine.

13. The lens system of claim 12, wherein the LEDs are mounted on the control board, and wherein each respective LED of the plurality of LEDs is individually-addressable via the electronic control signals.

14. The lens system of claim 12, wherein the control board includes an antenna operable for wirelessly receiving the electronic control signals from the light engine, such that the system is characterized by an absence of physical transfer conductors between the control board and the light engine.

15. The lens system of claim 12, wherein the control board is configured as a proximity sensor that is operable for detecting when the LEDs are separated from an external surface of the contact lens by a predetermined standoff distance, and for communicating an electronic proximity signal to the light engine that is indicative of the LEDs having reached the predetermined standoff distance.

16. The lens system of claim 12, wherein the LEDs have an adjustable setting that is responsive to the electronic control signals, and wherein the adjustable setting includes one or more of a color, a temperature, or a brightness level.

17. The lens system of claim 16, wherein the LEDs include a red, green, and blue diode LED array and at least one near-infrared LED.

18. A method for illuminating an interior of a patient's eye, comprising:
receiving electronic control signals from a light engine via a control board having an external light source, wherein the external light source is mounted to a surface of the control board;
illuminating the external light source in response to the electronic control signals from the light engine; and
emitting directed light from the external light source toward a contact lens arranged on the eye, the contact lens being configured to refract the directed light into the interior of the eye.

19. The method of claim 18, wherein emitting the directed light from the external light source toward the contact lens includes illuminating a prism array with the directed light, the prism array being connected to an annular tube containing the light source therein, and shaping the directed light into a conical distribution pattern using the prism array.

20. The method of claim 18, comprising:
detecting, via the control board, when the external light source is separated from an external surface of the contact lens by a predetermined standoff distance; and
transmitting an electronic proximity signal to the light engine that is indicative of the external light source having reached the predetermined standoff distance.
